(19) United States
(12) Patent Application Publication
(10) Pub. No.: US 2015/0038857 A1
(43) Pub. Date: Feb. 5, 2015

(54) BLOOD PRESSURE-RELATED INFORMATION DISPLAY DEVICE

(71) Applicants: OMRON Corporation, Kyoto-shi, Kyoto (JP); OMRON Healthcare Co., Ltd., Muko-shi, Kyoto (JP)

(72) Inventors: Chie Yamamoto, Kyoto (JP); Keisuke Yamada, Kyoto (JP); Mayumi Saito, Kyoto (JP); Naoki Tsuchiya, Kyoto (JP); Mitsuo Kuwabara, Kyoto (JP); Seiji Kono, Kyoto (JP); Osamu Shirasaki, Kyoto (JP); Hiroshi Ogawa, Kyoto (JP)

(73) Assignee: OMRON Corporation, Kyoto-shi, Kyoto (JP)

(21) Appl. No.: 14/385,287

(22) PCT Filed: Dec. 25, 2012
(86) PCT No.: PCT/JP2012/083508

§ 371 Date: Sep. 15, 2014

(30) Foreign Application Priority Data
Mar. 16, 2012 (JP) 2012-060276

(51) Int. Cl. A61B 5/021; A61B 5/00

(52) U.S. Cl. CPC A61B 5/021; A61B 5/7275
USPC 600/485

(57) ABSTRACT

A blood pressure-related information display device according to a preferred embodiment of the present invention displays, in a part of a display screen, a blood pressure evaluation item list display field indicating a plurality of types of blood pressure evaluation items and a quality display field indicating a quality of each of the blood pressure evaluation items as images. The blood pressure evaluation item list display field includes a header area and a body area. The blood pressure evaluation items are displayed in the body area in list format so all of the items can be viewed, and so that any one of the plurality of types of blood pressure evaluation items can be selected. Icon marks that indicate the quality of the blood pressure evaluation results are displayed in the quality display field, provided on the left side of the body area and arranged to the left of the corresponding blood pressure evaluation items.

### BACKGROUND OF THE PRESENT INVENTION

1. Field of the Present Invention

The present invention relates to blood pressure-related information display devices that display information related to a measurement subject's blood pressure in a display screen.

The present invention also relates to programs for causing a computer to execute a method for displaying information related to a measurement subject's blood pressure in a display screen.

### 2. Description of the Related Art

At present, a health management apparatus that includes an input means that inputs biological information, a setting means that sets a health management target value based on the biological information, and a memory means that records the biological information as history data, and that continuously evaluates the health management, is known as a device that displays information related to a measurement subject's blood pressure, as disclosed in JP 2003-288417A.

A data recording program that imports biological information into a personal computer through communication or manual input in order to support a measurement subject's health management is also known.

In addition, a health guidance support system is known, where when a health checkup examination result is inputted, examination items to which findings are to be added are specified based on a findings standard inputted in advance and are displayed in a display, as disclosed in JP 2003-216736A.

These systems (the apparatus, the program, and so on) support health-related guidance for the measurement subject based on biological information such as a blood pressure value.

However, the aforementioned conventional systems can only calculate a frequency of measurement, an average of measurement values, and so on for biological information measured over a given period. There is thus a problem in that it is difficult to make a more detailed evaluation of the biological information, and thus sufficient health-related guidance cannot be provided to the measurement subject. As a result, the measurement subject will not be very satisfied with the examination. Furthermore, even if detailed evaluation results could be obtained for the various types of biological information items to be evaluated, it is still difficult for a doctor to be aware of all evaluation results for a wide range of measurement subjects. There is thus a further problem in that when providing health-related guidance to a measurement subject, the doctor cannot quickly and easily select the evaluation items to be communicated to the measurement subject within the limited examination time, and thus cannot communicate effectively with the measurement subject.

### SUMMARY OF THE PRESENT INVENTION

Accordingly, preferred embodiment of the present invention provide a blood pressure-related information display device that displays information related to a measurement subject's blood pressure in a display screen, the device enabling a doctor to quickly and easily select blood pressure evaluation items to be communicated to a measurement subject from a variety of blood pressure evaluation items, and thus enabling the doctor to communicate effectively with the measurement subject as well as increasing the measurement subject's level of satisfaction with an examination.

Preferred embodiments of the present invention also provide a non-transitory computer-readable medium including a computer program that causes a computer to execute a method for displaying information related to a measurement subject's blood pressure in a display screen, the device enabling a doctor to quickly and easily select blood pressure evaluation items to be communicated to a measurement subject from a variety of blood pressure evaluation items, and thus enabling the doctor to communicate effectively with the measurement subject as well as increasing the measurement subject's level of satisfaction with an examination.

A blood pressure-related information display device according to a preferred embodiment of the present invention displays information related to a measurement subject's blood pressure in a display screen, and includes a blood pressure data obtainment unit configured to obtain blood pressure data of the measurement subject, an evaluation result calculation unit configured to calculate, for a predetermined plurality of blood pressure evaluation items used to evaluate a state of the measurement subject's blood pressure, a blood pressure evaluation result indicating a quality of each of the plurality of blood pressure evaluation items by executing a statistical process on the blood pressure data measured on each day throughout a given period and evaluating a change in the blood pressure over time, and a display control unit configured or programmed to perform control to display a blood pressure evaluation item list enabling the plurality of blood pressure evaluation items to be viewed as a list in the display screen and displaying the calculated blood pressure evaluation results in association with and next to corresponding ones of the plurality of blood pressure evaluation items in the blood pressure evaluation item list.

In the present specification, “blood pressure data” refers generally to a systolic blood pressure (SBP), which is a maximum blood pressure, a diastolic blood pressure (DBP), which is a minimum blood pressure, as well as a pulse frequency, for example.

“Blood pressure evaluation result”, meanwhile, refers to a result of evaluating the blood pressure data, for example.

With the blood pressure-related information display device according to a preferred embodiment of the present invention, first, the blood pressure data obtainment unit obtains the blood pressure data from a measurement subject (typically a patient; the same applies hereinafter). The evaluation result calculation unit calculates, for a predetermined plurality of blood pressure evaluation items used to evaluate a state of the measurement subject's blood pressure, a blood pressure evaluation result indicating a quality of each of the plurality of blood pressure evaluation items by executing a statistical process on the blood pressure data measured on each day throughout a given period and evaluating a change in the blood pressure over time. The display control unit displays the blood pressure evaluation item list enabling the plurality of blood pressure evaluation items to be viewed as a list in the display screen. The display control unit also is configured or programmed to perform control to display the calculated blood pressure evaluation results in association with and next to corresponding ones of the plurality of blood pressure evaluation items in the displayed blood pressure evaluation item list.

According to this blood pressure-related information display device, when the device is operating in this manner, a doctor, for example, can view the blood pressure evaluation results displayed in association with and next to corresponding ones of the plurality of blood pressure evaluation items using the blood pressure evaluation item list, which is displayed in the display screen. Accordingly, the doctor can select, from the blood pressure evaluation items, a blood pressure evaluation item that enables effective communication with the measurement subject in a short amount of time by referring to the quality of the blood pressure evaluation results for the plurality of blood pressure evaluation items in the blood pressure evaluation item list, which increases the measurement subject's level of satisfaction with the examination and reduces the amount of time required for the examination.

In the blood pressure-related information display device according to another preferred embodiment of the present invention, the blood pressure evaluation result is displayed as a mark that is distinguishable by color or by shape.

In the blood pressure-related information display device according to this preferred embodiment of the present invention, the blood pressure evaluation result is displayed as a mark that is distinguishable by color or by shape. Accordingly, the doctor can refer to the blood pressure evaluation item list in the display screen and can select, from the blood pressure evaluation items, an evaluation item that enables effective communication with the measurement subject in a shorter amount of time and with certainty.

A blood pressure-related information display device according to another preferred embodiment of the present invention further includes a comment storage unit configured to store a comment for the quality, an item selection unit configured to select the blood pressure evaluation item for which the quality is displayed from the blood pressure evaluation item list, and a comment display control unit configured or programmed to perform control to display, in the display screen, a comment regarding the quality of the selected blood pressure evaluation item by referring to the comment storage unit.

In the blood pressure-related information display device according to this preferred embodiment, a comment regarding the aforementioned quality is displayed in the display screen for the selected blood pressure evaluation item. Accordingly, the doctor can view not only the aforementioned quality but also the comment regarding the quality for the blood pressure evaluation item whose quality is displayed, in the display screen. Accordingly, the doctor can communicate effectively with the measurement subject, increasing the measurement subject's level of satisfaction with the examination, while also enabling the examination to be carried out in a shorter amount of time.

In the blood pressure-related information display device according to another preferred embodiment of the present invention, the comment storage unit is configured to store the comment in accordance with content of the quality.

In the blood pressure-related information display device according to this preferred embodiment, the comment is stored in accordance with the content of the quality, and thus the doctor can refer to the comment based on the content of the quality in the display screen. Accordingly, the doctor can communicate effectively with the measurement subject, increasing the measurement subject's level of satisfaction with the examination, while also enabling the examination to be carried out in a shorter amount of time.

A non-transitory computer-readable medium including a computer program according to another preferred embodiment of the present invention causes a computer to execute a method for displaying information related to a measurement subject's blood pressure in a display screen, the method including a first step of obtaining blood pressure data of the measurement subject, a second step of calculating, for a predetermined plurality of blood pressure evaluation items used to evaluate a state of the measurement subject's blood pressure, a blood pressure evaluation result indicating a quality of each of the plurality of blood pressure evaluation items by executing a statistical process on the blood pressure data measured on each day throughout a given period and obtained in the first step and evaluating a change in the blood pressure over time, and a third step of displaying a blood pressure evaluation item list enabling the plurality of blood pressure evaluation items to be viewed as a list in the display screen and displaying the calculated blood pressure evaluation results in association with and next to corresponding ones of the plurality of blood pressure evaluation items in the displayed blood pressure evaluation item list.

With the non-transitory computer-readable medium including a non-transitory computer-readable medium including a computer program according to this preferred embodiment of the present invention, a computer is caused to execute a method for displaying information related to a measurement subject's blood pressure in a display screen in the following manner. That is, in the first step, blood pressure data of the measurement subject is obtained. In the second step, a blood pressure evaluation result indicating the quality of each of the blood pressure evaluation items, is calculated for a plurality of the predetermined blood pressure evaluation items used to evaluate a state of the measurement subject's blood pressure by executing a statistical process on the blood pressure data measured on each day throughout a given period and obtained in the first step and evaluating a change in the blood pressure data over time. In the third step, a blood pressure evaluation item list enabling the plurality of blood pressure evaluation items to be viewed is displayed as a list in the display screen and the calculated blood pressure evaluation results are displayed in association with and next to corresponding ones of the plurality of blood pressure evaluation items in the displayed blood pressure evaluation item list.

In the case where a computer is caused to execute such a method, for example, the doctor can view the blood pressure evaluation results displayed next to the associated blood pressure evaluation items in the display screen, using the blood pressure evaluation item list. Accordingly, the doctor can select, from the blood pressure evaluation items, a blood pressure evaluation item that enables effective communication with the measurement subject in a short amount of time by referring to the blood pressure evaluation item list, which increases the measurement subject's level of satisfaction with the examination and reduces the amount of time required for the examination.

As is clear from the above descriptions, according to the blood pressure-related information display device and non-transitory computer-readable medium including a non-transitory computer-readable medium including a computer program according to various preferred embodiments of the present invention, a doctor can quickly and easily select blood pressure evaluation items to be communicated to a measurement subject from a variety of blood pressure evaluation items, thus enabling the doctor to communicate effectively with the measurement subject as well as increasing the measurement subject's level of satisfaction with an examination.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a block diagram illustrating a preferred embodiment of the present invention in which a blood pressure-related information display device is configured as a system on a network.

FIG. 2 is a block diagram illustrating the configuration of a hospital terminal that is included in the system of FIG. 1.

FIG. 3 is a block diagram illustrating the configuration of a server that is included in the system of FIG. 1.

FIG. 4 is a block diagram illustrating the configuration of a blood pressure meter that is included in the system of FIG. 1.

FIG. 5 is a diagram illustrating an overview of a flow of operations performed by the server.

FIG. 6 is a diagram illustrating values of changes from a representative value in an initial examination to a representative value in a current examination.

FIG. 7A is a diagram illustrating a correspondence between determination values and determination segments for representative values of blood pressures measured in the morning.

FIG. 7B is a diagram illustrating a correspondence between determination values and determination segments for representative values of blood pressures measured in the evening.

FIG. 8 is a diagram illustrating correspondence between determination segments, comments, and a quality of the comments.

FIG. 9 is a diagram illustrating an overview of a flow of operations performed by the hospital terminal.

FIG. 10 is a diagram illustrating an example in which fluctuations in a systolic blood pressure value and a diastolic blood pressure value, measured at mornings and evenings from approximately 60 days prior to a current examination day, are displayed as images in a display screen of the hospital terminal.

FIG. 11 is a diagram illustrating an example in which a blood pressure evaluation item list is displayed as an image in the display screen of the hospital terminal.

FIG. 12 is a diagram illustrating an example in which a graph indicating changes in a blood pressure level from an initial level is displayed along with comments as an image in the display screen of the hospital terminal.

FIG. 13 is a diagram illustrating an overview of a flow of operations performed by the hospital terminal when functionality of the server is incorporated into the hospital terminal.

### DETAILED DESCRIPTION OF THE PREFERRED EMBODIMENTS

Hereinafter, preferred embodiments of the present invention will be described in detail with reference to the drawings.

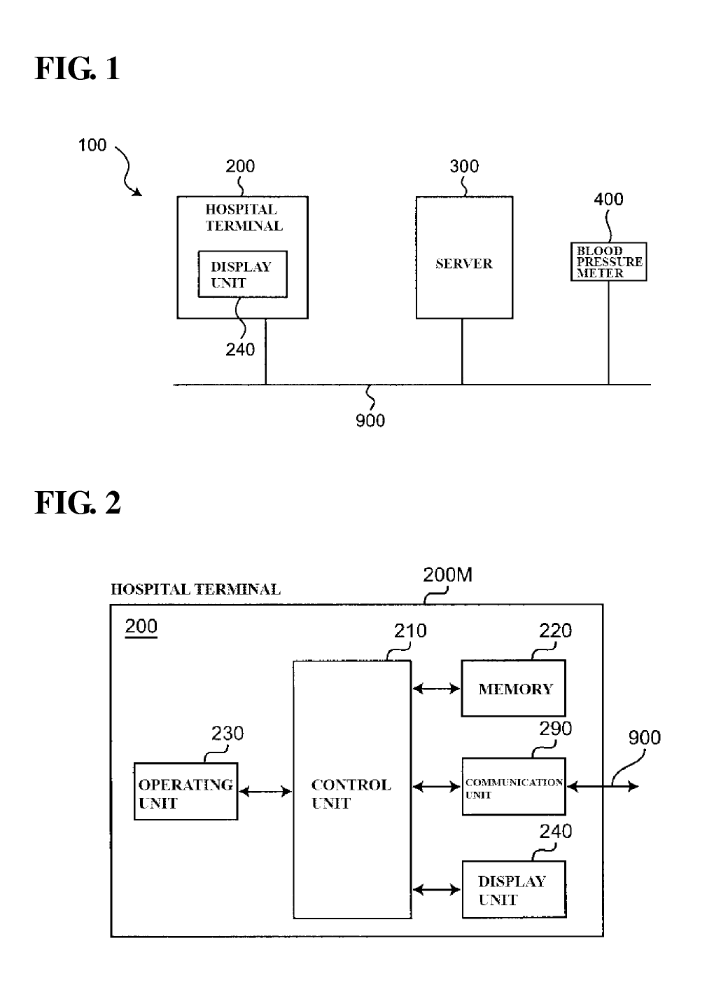

FIG. 1 illustrates an example in which a blood pressure-related information display device according to a preferred embodiment of the present invention preferably is embodied as a system on a network (indicated overall by reference numeral 100). The system 100 includes a hospital terminal 200 including a display unit 240 defining and serving as a display screen, a server 300, and a blood pressure meter 400. The hospital terminal 200, the server 300, and the blood pressure meter 400 are configured to communicate with each other over a network 900.

As shown in FIG. 2, the hospital terminal 200 includes a main body 200M, as well as a control unit 210, a memory 220, an operating unit 230, the display unit 240 defining and serving as a display screen, and a communication unit 290, which are provided in the main body 200M. The hospital terminal 200 is configured of a commercially-available laptop computer in which application software (computer programs) to execute processes that will be described later is installed, and is capable of accessing the server 300.

The control unit 210 includes a central processing unit (CPU) as well as auxiliary circuitry thereof, and is configured or programmed to control the various units of the hospital terminal 200, and execute processes that will be described later in accordance with programs and data stored in the memory 220. In other words, the control unit 210 is configured or programmed to process data inputted through the operating unit 230 and the communication unit 290, and stores the processed data in the memory 220, display the processed data in the display unit 240, output the processed data from the communication unit 290, or the like.

The memory 220 includes a random access memory (RAM) used as a work region required by the control unit 210 to execute programs, and a read-only memory (ROM) configured to store basic programs to be executed by the control unit 210. A semiconductor memory (a memory card, a solid-state drive (SSD)) or the like may be used as a storage medium in an auxiliary storage unit to complement a storage region in the memory 220.

The operating unit 230 is in this example preferably configured of a keyboard and a mouse, and inputs operation signals indicating operations performed by a user to a control unit 310. Instead of or in addition to the keyboard and the mouse, the operating unit 230 may be configured of another operating device, such as a touch panel.

The display unit 240 includes a display screen (for example, a liquid-crystal display (LCD) or an electroluminescence (EL) display). The display unit 240 displays a predetermined image in the display screen under the control of the control unit 210.

The communication unit 290 sends information from the control unit 210 to the server 300 via the network 900.

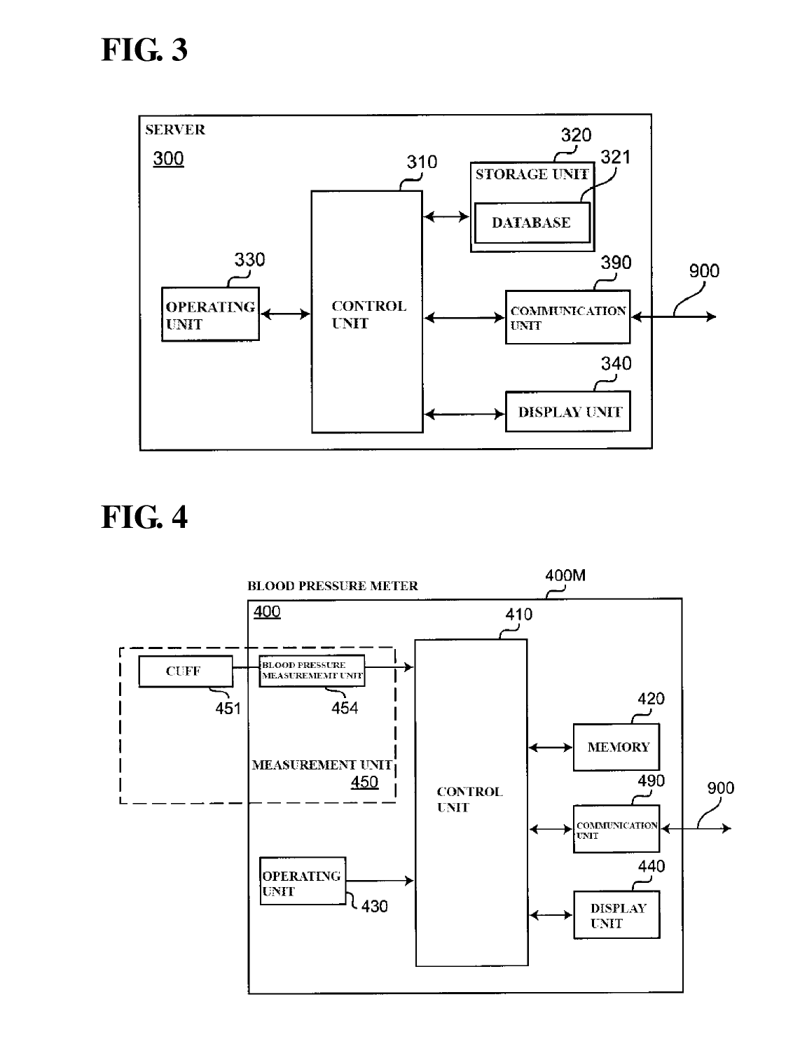

As shown in FIG. 3, the server 300 includes the control unit 310, a storage unit 320, an operating unit 330, a display unit 340, and a network communication unit 390. The server 300 is realized by installing programs (software) to execute processes that will be mentioned later in a generic computer device.

The control unit 310 includes a CPU and auxiliary circuitry thereof. The control unit 310 is configured or programmed to control the various units of the server 300, execute predetermined processes in accordance with programs and data stored in the storage unit 320, process data inputted from the operating unit 330 and the communication unit 390, store processed data in the storage unit 320, display processed data in the display unit 340, and output processed data from the communication unit 390.

The storage unit 320 includes a RAM used as a work region required by the control unit 310 to execute programs, and a ROM configured to store basic programs to be executed by the control unit 310. A database 321 containing blood pressure measurement data sent from many users is provided in the storage unit 320. A magnetic disk (a hard disk (HD), a flexible disk (FD)), an optical disc (a compact disc (CD), a digital versatile disc (DVD), a Blu-ray disc (BD)), a magneto-optical disc (MO), or a semiconductor memory (a memory card, a solid state drive (SSD)), or the like may be used as a storage medium in an auxiliary storage unit to complement the storage region of the storage unit 320.

The operating unit 330 is in this example preferably configured of a keyboard and a mouse, and inputs operation signals indicating operations performed by a user to a control unit 310. Instead of or in addition to the keyboard and the mouse, the operating unit 330 may be configured of another operating device, such as a touch panel.

The display unit 340 includes a display screen (for example, an LCD or an EL display). The display unit 340 displays a predetermined image in the display panel under the control of the control unit 310.

The network communication unit 390 sends information from the control unit 310 to other apparatuses (in this example, the hospital terminal 200) via the network 900, and receives information sent via the network 900 from another apparatus and passes the information to the control unit 310.

As shown in FIG. 4, the blood pressure meter 400 includes a casing 400M, as well as a control unit 410, a memory 420, an operating unit 430, a display unit 440, a measurement unit 450 defining and serving as a blood pressure data obtainment unit, and a communication unit 490, which are provided in the casing 400M.

In response to an operational device such as an operation button, an operation key, or the like (not shown) being manipulated by an operator, the operating unit 430 inputs operation signals based on the details of the operation to the control unit 410.

The measurement unit 450 includes a cuff 451 and a blood pressure measurement unit 454 configured to measure a blood pressure, and obtains measured values of a systolic blood pressure (SBP), a diastolic blood pressure (DBP), and a pulse frequency from a patient.

The memory 420 stores data of programs to control the blood pressure meter 400, configuration data to configure various types of functions of the blood pressure meter 400, measurement result data, and so on. The memory 420 is also used as a working memory when programs are executed.

The control unit 410 includes a CPU, and controls the memory 420, the display unit 440, and the communication unit 490 in accordance with programs stored in the memory 420 to control the blood pressure meter 400, in response to operation signals from the operating unit 430, and based on detection signals from the measurement unit 450.

The display unit 440 includes a display panel, indicators, and the like, and displays predetermined information in accordance with control signals from the control unit 410.

Under the control of the control unit 410, the communication unit 490 sends predetermined information to an external apparatus over the network 900, receives information from an external apparatus over the network 900 and passes the information to the control unit 410, and so on. The communication over the network 900 may be wireless or wired. Although the network 900 preferably is the Internet in this preferred embodiment, the network 900 is not limited thereto, and may be another type of network such as a local area network (LAN) installed in a hospital, one-to-one communication using a USB cable, or the like.

The system 100 is preferably used as follows.

i) Using the blood pressure meter 400, the measurement subject measures his/her own blood pressure at home, for example, in the morning and evening of each day throughout a given period. Once the measurement is finished, the blood pressure meter 400 sends the measured blood pressure data to the server 300 via the network 900 along with a date/time of the measurement.

ii) Upon receiving the blood pressure data from the blood pressure meter 400 while in an operational state, the server 300 stores the received blood pressure data in the storage unit 320 along with the measurement date/time.

iii) When a doctor operates the operating unit 230 of the hospital terminal 200 while the hospital terminal 200 is in an operational state and selects “blood pressure evaluation display”, the control unit 210 of the hospital terminal 200 issues, to the server 300, a request to obtain a blood pressure evaluation result along with sending date data indicating a current examination date.

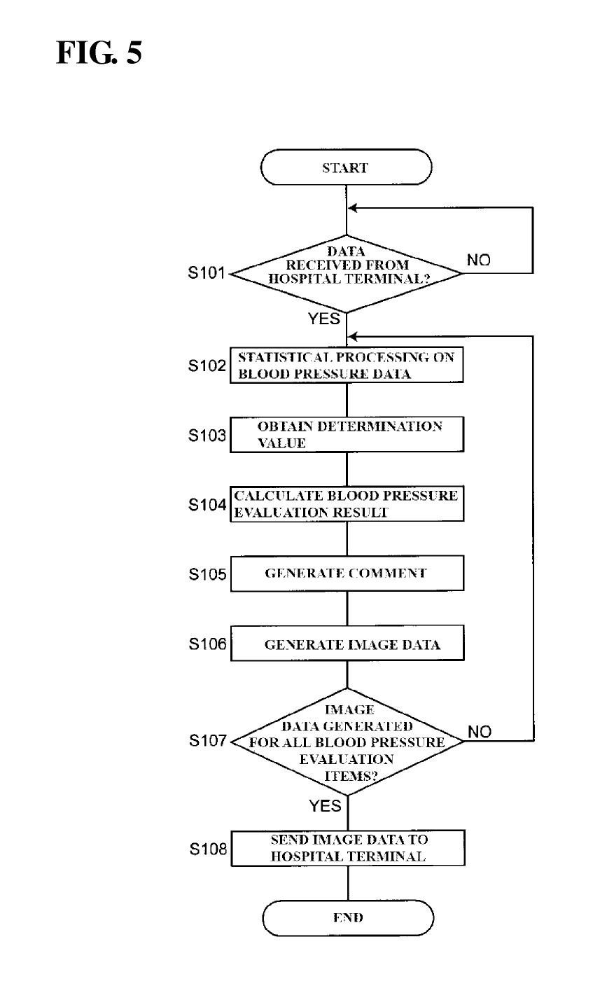

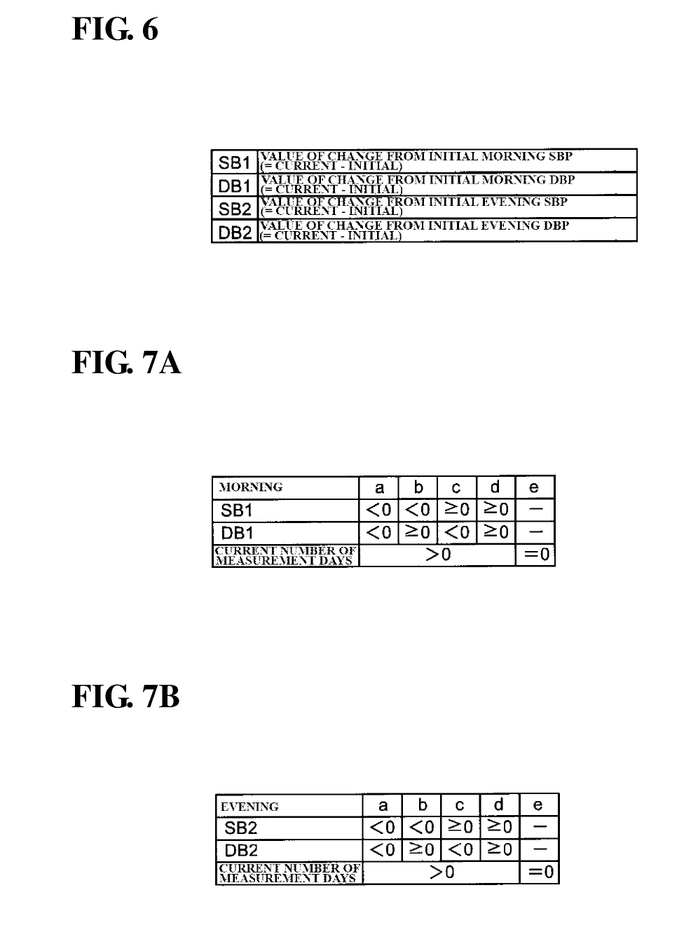

iv) With the server 300 in an operational state, the control unit 310 of the server 300 stands by for the data including the date data to be sent from the hospital terminal 200 via the network 900, as indicated by step S101 in FIG. 5. Upon receiving the data from the hospital terminal 200 over the network 900 via the communication unit 390 (YES in step S101), the control unit 310 operates as an evaluation result calculation unit, and performs statistical processing on the stored blood pressure data for a predetermined blood pressure evaluation item (step S102).

“Change in blood pressure value over time”, “number of measurement days per week”, “measurement frequency per day of the week”, “change in morning-evening average/morning-evening difference (SBP)”, “occurrence of tachycardia/bradycardia”, “change in blood pressure level from initial value”, “average blood pressure value (by day of the week)”, “change in blood pressure distribution (SBP)”, and “change in blood pressure distribution (DBP)” can be given as examples of the stated predetermined blood pressure evaluation items.

Specifically, in the case where the “change in blood pressure level from initial value” is to be statistically processed (the same applies hereinafter), the control unit 310 calculates representative values, for both the morning and evening, from the current examination, the previous examination, the second-to-last examination, and the initial examination.

In other words, based on the measurement subject's blood pressure value measured during a period from the previous examination to the current examination (a current examination period) and a number of measurement days, the control unit 310 calculates an average of the blood pressure values in the current examination period as a representative value for the current examination. Likewise, an average of the blood pressure values in a period from the second-to-last examination to the previous examination (a previous examination period) is calculated as a representative value for the previous examination. Furthermore, an average of the blood pressure values in a period spanning from three examinations prior to the current examination up to the second-to-last examination (a second-to-last examination period) is calculated as a representative value for the second-to-last examination. Finally, an average of the blood pressure values in a period from when the blood pressure meter 400 was first used to take a measurement to the first examination (an initial examination period) is calculated as a representative value for the initial examination.

In this example, the representative value for the current examination (a set of the SBD and the DBP is indicated by SBP/DBP; the same applies hereinafter) preferably is approximately 145/78 mmHg in both the morning and the evening. The representative value for the previous examination preferably is approximately 162/103 mmHg in the morning and approximately 162/102 mmHg in the evening, whereas the representative value for the second-to-last examination preferably is approximately 177/122 mmHg in the morning and approximately 178/122 mmHg in the evening. Furthermore, the representative value for the initial examination preferably is approximately 190/135 mmHg in both the morning and the evening. Note that the aforementioned number of measurement days in the current examination period preferably is 5 days for the morning and 10 days for the evening.

Next, operating as the evaluation result calculation unit, the control unit 310 obtains a determination value to calculate the blood pressure evaluation result from the statistically-processed blood pressure data (step S103).

Specifically, to use the above example, the control unit 310 calculates a value of change from the representative value in the initial examination to the representative value in the current examination along with a number of days in which the blood pressure has been measured in the current examination period, for both the SBP and the DBP in both the morning and the evening, and uses the result of this calculation as the determination value.

In other words, for both the SBP and the DBP in the morning, when the stated values of change are represented by SB1 and DB1 and the number of measurement days is represented by D1, the following holds true: SB1=current−initial (SBP)=145−190=−45<0; DB1=current−initial (DBP)=78−135=−57<0; and D1=number of days in which the blood pressure has been measured in the current examination period=10>0.

On the other hand, for both the SBP and the DBP in the evening, when the stated values of change are represented by SB2 and DB2 and the number of measurement days is represented by D2, the following holds true: SB2=current−initial (SBP)=145−190=−45<0; DB2=current−initial (DBP)=78−135=−57<0; and D2=number of days in which the blood pressure has been measured in the current examination period=5>0.

Next, operating as the evaluation result calculation unit, the control unit 310 calculates a quality as the blood pressure evaluation result for each of the stated blood pressure evaluation items, based on the obtained determination value (step S104).

Specifically, to use the above example, the control unit 310 obtains determination segments, as indicated in FIGS. 7A and 7B, based on the stated determination value. In other words, the control unit 310 refers to FIG. 7A and obtains a determination segment “a” based on the aforementioned determination value for the morning, or in other words, based on the values of change SB1 and DB1 and the number of measurement days D1. Likewise, the control unit 310 refers to FIG. 7B and obtains a determination segment “a” based on the aforementioned determination value for the evening, or in other words, based on the value of change SB2,DB2 and the number of measurement days D2.

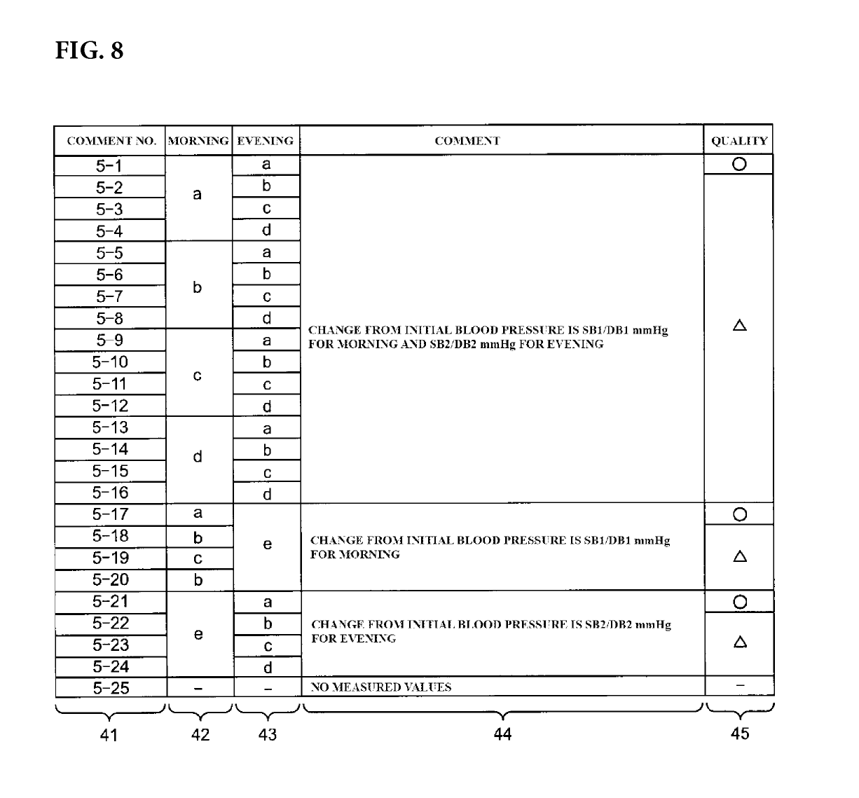

Then, based on the obtained morning determination segment “a” and evening determination segment “a”, the control unit 310 refers to FIG. 8, which indicates correspondences between the determination segments, comments, and qualities, and obtains “∘”, indicating “good”, as a result.

Next, operating as a comment generating unit, the control unit 310 generates a comment to be displayed in the display unit 240 of the hospital terminal 200 based on the obtained determination value (step S105 in FIG. 5).

Specifically, to use the above example, the control unit 310 uses the determination value and the obtained determination segment and refers to FIG. 8, which indicates corresponding comments, and generates a comment corresponding to Comment No. 5-1, which reads “change from initial blood pressure is −45/−57 mmHg for morning and −45/−57 mmHg for evening”.

FIG. 8 includes a Comment No. column 41, a determination segment column 42 indicating morning determination segments, a determination segment column 43 indicating evening determination segments, a comment column 44 indicating the content of comments, and a quality column 45 indicating quality determination results. Based on combinations of the morning determination segments “a”, “b”, “c”, “d”, and “e” and the evening determination segments “a”, “b”, “c”, “d”, and “e”, the corresponding comment is selected from the comment column 44, and “∘”, indicating “good”, or “Δ”, indicating “poor”, is selected from the quality column 45.

Next, based on the statistically-processed blood pressure data, the blood pressure evaluation result, and the comment, the control unit 310 generates image data (which will be described later) to be displayed in the display unit 240 of the hospital terminal 200 (step S106 in FIG. 5).

Then, when the image data and comments have been generated for all of the blood pressure evaluation items (YES in step S107), the control unit 310 stores the generated image data in the database 321. The image data and comment are also sent from the communication unit 390 to the hospital terminal 200 over the network 900 (step S108).

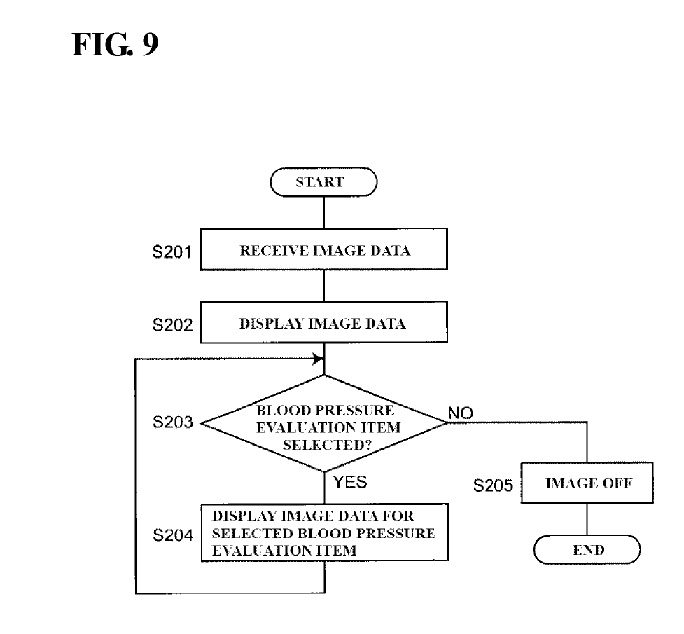

v) As indicated by the flow shown in FIG. 9, the hospital terminal 200 receives the image data from the network 900 via the communication unit 290 (step S201) and, causing the memory 220 to function as an image data storage unit and a comment storage unit, stores the image data and the comment. Next, operating as a display control unit, the control unit 210 of the hospital terminal 200 displays, in the display screen of the display unit 240, the image data stored in the memory 220 (step S202).

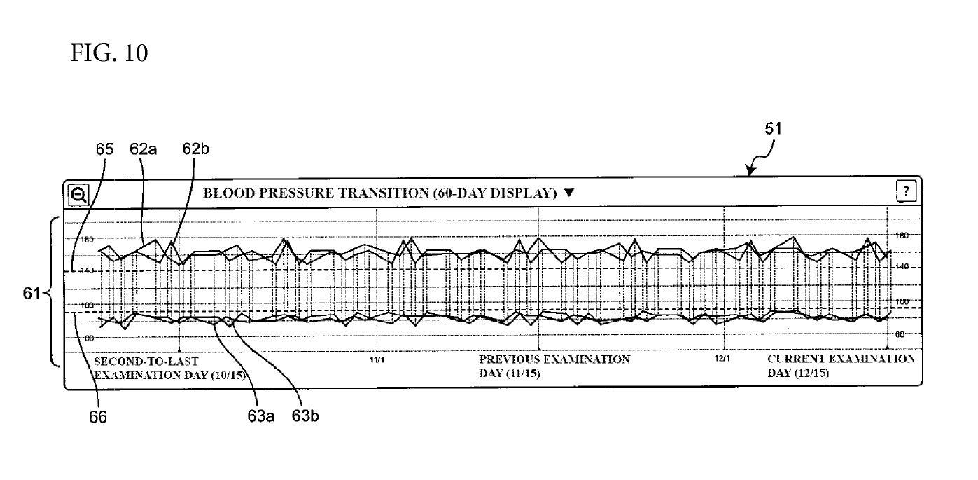

The image displayed in a part 51 of the display screen of the display unit 240 at this stage is as illustrated in FIG. 10, for example. As shown in FIG. 10, a blood pressure transitive graph display field 61, indicating the change in blood pressure value over time for the measurement subject, is displayed as an image.

In the blood pressure transitive graph display field 61, the measurement subject's blood pressure values for each day corresponding to an approximately 60-day period previous to the current examination day (December 15 (December 15), in this example) are displayed as polygonal line graphs 62a, 62b, 63a, and 63b, with the horizontal axis representing time (in days) and the vertical axis representing the blood pressure value (in mmHg).

Here, the polygonal line 62a indicates the SBP measured in the morning, whereas the polygonal line 62b indicates the SBP measured in the evening. Likewise, the polygonal line 63a indicates the DBP measured in the morning, whereas the polygonal line 63b indicates the DBP measured in the evening. These polygonal line graphs 62a, 62b, 63a, and 63b are generated through the aforementioned statistical process (and in particular, through step S102 in FIG. 5). Accordingly, using the display unit 240, the doctor can instinctively understand the change in blood pressure value over time for the measurement subject in the approximately 60 days prior to the current examination day (December 15).

The blood pressure transitive graph display field 61 also includes broken lines 65 and 66 that extend straight in the horizontal direction. The broken line 65 indicates about 140 mmHg as a target value for the SBP set by the measurement subject or a doctor in charge, whereas the broken line 66 indicates about 90 mmHg as a target value for the DBP set by the measurement subject or the doctor in charge, for example. Accordingly, by comparing the polygonal line graphs 62a, 62b, 63a, and 63b to the broken lines 65 and 66, the doctor can determine at a glance whether or not the measurement subject's blood pressure values have met the target values set as described above.

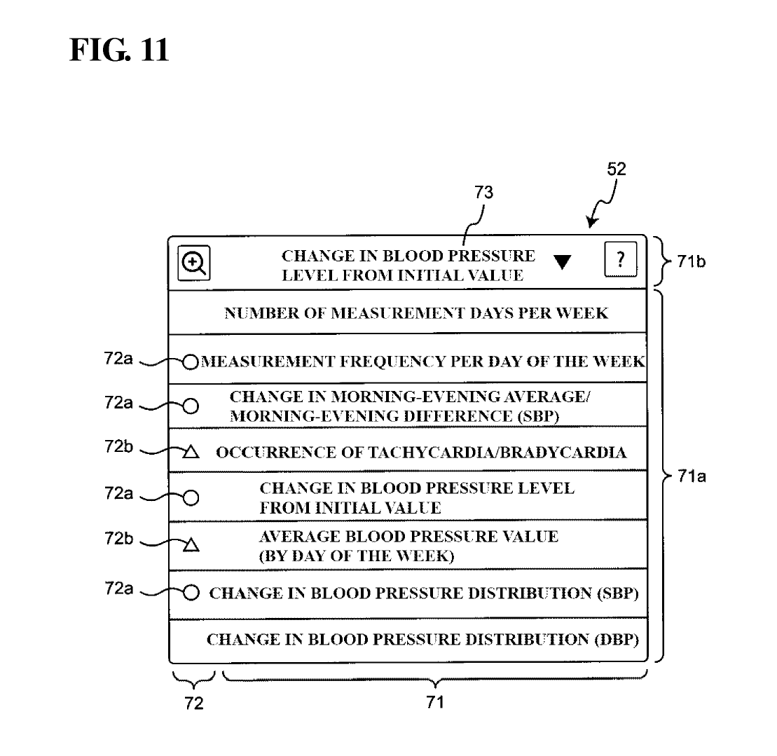

The image displayed in a portion 52 of the display screen of the display unit 240 at this stage is as illustrated in FIG. 11, for example. As shown in FIG. 11, a blood pressure evaluation item list display field 71, indicating a plurality of types of the blood pressure evaluation items, and a quality display field 72, indicating the quality for each of the blood pressure evaluation items, are displayed as images.

The blood pressure evaluation item list display field 71 includes a header area 71b and a body area 71a, in that order from the top.

The blood pressure evaluation items are displayed in the body area 71a in list format so that all the items can be viewed. In other words, “number of measurement days per week”, “measurement frequency per day of the week”, “change in morning-evening average/morning-evening difference (SBP)”, “occurrence of tachycardia/bradycardia”, “change in blood pressure level from initial value”, “average blood pressure value (by day of the week)”, “change in blood pressure distribution (SBP)”, and “change in blood pressure distribution (DBP)” are displayed, in that order from top to bottom, as the blood pressure evaluation items.

The body area 71a enables one of the plurality of types of blood pressure evaluation item to be selected.

A blood pressure evaluation item 73 selected in the body area 71a is displayed in the header area 71b (in this example, “change in blood pressure level from initial value” is selected in the body area 71a and displayed), and the selected blood pressure evaluation item 73 is capable of being selected further.

Icon marks 72a and 72b that indicate the quality of the blood pressure evaluation results are displayed in the quality display field 72, provided on the left side of the body area 71a and arranged to the left of the corresponding blood pressure evaluation items. The icon mark 72a is circular, indicating that the blood pressure evaluation result is “good”. On the other hand, the icon mark 72b is triangular, indicating that the blood pressure evaluation result is “poor”. In this example, of the blood pressure evaluation items, the icon mark 72a is displayed for the “measurement frequency per day of the week”, “change in morning-evening average/morning-evening difference (SBP)”, “change in blood pressure level from initial value”, and “change in blood pressure distribution (SBP)”. On the other hand, of the blood pressure evaluation items, the icon mark 72b is displayed for the “occurrence of tachycardia/bradycardia” and “average blood pressure value (by day of the week)”.

In this manner, the doctor can refer to the icon marks 72a and 72b in the portion 52 of the display screen and select, from the blood pressure evaluation item list display field 71, an evaluation item that enables more useful communication with the measurement subject, with more certainty and in a shorter amount of time.

Although the icon marks 72a and 72b are described here as having different shapes, the present invention is not limited thereto, and the marks may be displayed having different colors to make the marks identifiable.

Next, it is assumed that the doctor selects the header area 71b, or in other words, selects the selected blood pressure evaluation item 73 (YES in step S203 of FIG. 9). Upon doing so, operating as the display control unit and a comment display control unit, the control unit 210 displays image data regarding the blood pressure evaluation result for the selected blood pressure evaluation item 73 in the portion 52 of the display screen of the display unit 240 (step S204 in FIG. 9).

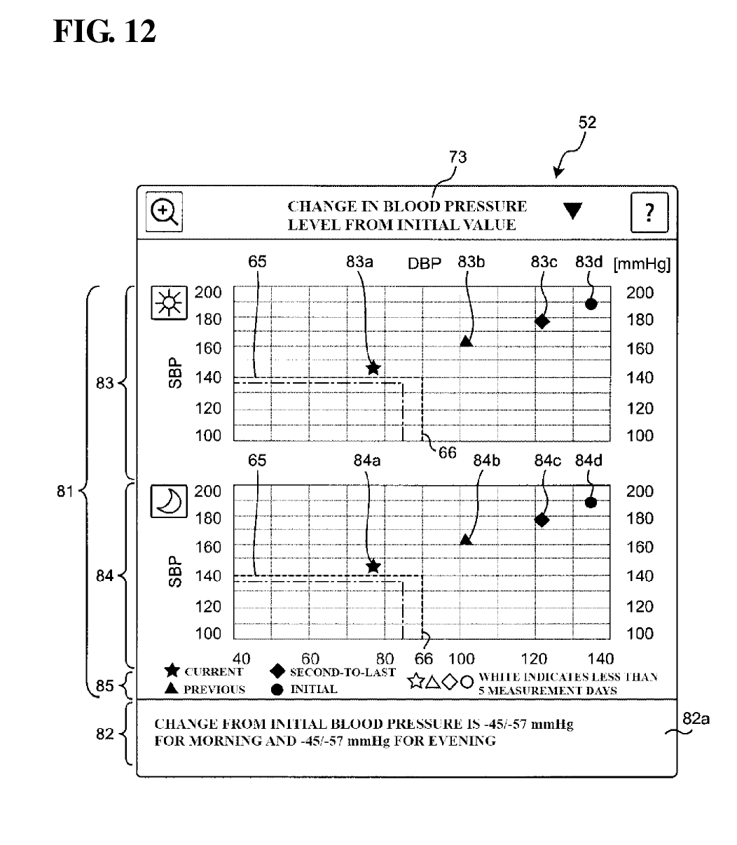

The image displayed in the portion 52 of the display screen of the display unit 240 at this stage is as illustrated in FIG. 12, for example. Specifically, a blood pressure level graph display field 81 and a comment display field 82 are displayed as images.

The blood pressure level graph display field 81 includes a morning blood pressure level graph display field 83, an evening blood pressure level graph display field 84, and a notes field 85.

In the morning blood pressure level graph display field 83, the measurement subject's morning representative values for the current examination, the previous examination, the second-to-last examination, and the initial examination are plotted as points 83a, 83b, 83c, and 83d, with the horizontal axis representing DBP (in mmHg) and the vertical axis representing the SBP (in mmHg).

Likewise, in the evening blood pressure level graph display field 84, the measurement subject's evening representative values for the current examination, the previous examination, the second-to-last examination, and the initial examination are plotted as points 84a, 84b, 84c, and 84d, with the horizontal axis representing DBP (in mmHg) and the vertical axis representing the SBP (in mmHg).

Furthermore, a comment 82a in response to the value of change from the representative values 83d and 84d for the initial examination to the representative values 83a and 84a for the current examination (reading “change from initial blood pressure is −45/−57 mmHg for the morning and −45/−57 mmHg for the evening”, in this example) is displayed in the comment display field 82.

Accordingly, the doctor can view not only the aforementioned quality but also the comment 82a regarding the quality for each blood pressure evaluation item whose quality is displayed, in the portion 52 of the display screen. Accordingly, the doctor can communicate effectively with the measurement subject, increasing the measurement subject's level of satisfaction with the examination, while also enabling the examination to be carried out in a shorter amount of time.

When no operations have been made for a set amount of time (60 seconds, for example) while the image shown in FIG. 12 is displayed in the portion 52 of the display screen of the display unit 240 (NO in step S203 of FIG. 9), the control unit 210 turns the image off in order to conserve energy (step S205 in FIG. 9).

Through this, the doctor can view a list of the blood pressure evaluation items displayed with associated blood pressure evaluation results in the portion 52 of the display screen, using the blood pressure evaluation item list display field 71. Accordingly, the doctor can select, from the blood pressure evaluation items, a blood pressure evaluation item that enables effective communication with the measurement subject in a short amount of time by referring to the blood pressure evaluation item list display field 71, which increases the measurement subject's level of satisfaction with the examination and reduces the amount of time required for the examination.

Note that the control unit 310 of the server 300 may send only data to generate an image to the hospital terminal 200, rather than sending the image data, and the control unit 210 of the hospital terminal 200 may then generate the image itself.

Furthermore, although the blood pressure-related information display device according to a preferred embodiment of the present invention is configured of a system 100 preferably including a hospital terminal 200, a server 300, and a blood pressure meter 400 connected over a network in this preferred embodiment, the present invention is not limited thereto.

For example, the blood pressure-related information display device according to a preferred embodiment of the present invention may be configured of only the blood pressure meter 400 and the hospital terminal 200. That is, in addition to the functions originally performed by the control unit 210, memory 220, the operating unit 230, the display unit 240, and the communication unit 290 in the hospital terminal 200, these units preferably are configured to also carry out the functions of the control unit 310, the storage unit 320, the operating unit 330, the display unit 340, and the network communication unit 390 in the server 300, respectively. In this case, a non-transitory computer-readable medium including a computer program that causes the control unit 210 to execute a method that displays information related to the measurement subject's blood pressure in the display screen, as will be described below, is installed in the memory 220 of the hospital terminal 200. Through this, the blood pressure-related information display device according to a preferred embodiment of the present invention can be configured as a small-sized, compact unit.

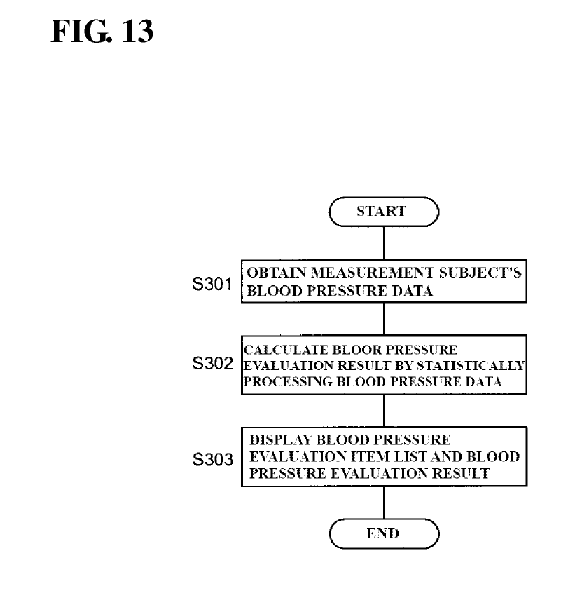

The stated method can be broadly divided into three steps, as indicated in FIG. 13.

In a first step S301 shown in FIG. 13, the measurement subject's blood pressure data, measured by the blood pressure meter 400, is obtained.

Ina second step S302, the blood pressure evaluation results are calculated for the predetermined blood pressure evaluation items used to evaluate the measurement subject's blood pressure state, by executing statistical processing on the blood pressure data measured each day throughout a given period and obtained in the aforementioned first step S301. This second step S302 corresponds to steps S102 to S104 in FIG. 5.

In a third step S303, the blood pressure evaluation item list display field 71 that enables the blood pressure evaluation items to be viewed as a list is displayed, along with the icon marks 72a and 72b that indicate the quality of the calculated blood pressure evaluation results in correspondence with the blood pressure evaluation items in the displayed blood pressure evaluation item list, in the portion 52 of the display screen. This third step S303 corresponds to step S202 in FIG. 9.

In the case where a computer is caused to execute such a method, for example, the doctor can view a list of the blood pressure evaluation items displayed with associated blood pressure evaluation results in the portion 52 of the display screen, using the blood pressure evaluation item list display field 71. Accordingly, the doctor can select, from the blood pressure evaluation items, a blood pressure evaluation item that enables effective communication with the measurement subject in a short amount of time by referring to the blood pressure evaluation item list display field 71, which increases the measurement subject's level of satisfaction with the examination and reduces the amount of time required for the examination.

The non-transitory computer-readable medium including a computer program can be a recording medium such as a CD, a DVD, a flash memory, or the like as application software. By being installed in what is substantially a computer device, such as a hospital terminal, a personal computer, a personal digital assistant (PDA), a smartphone, or the like, the application software recorded onto the recording medium can cause the computer device to execute the method for displaying the information related to the measurement subject's blood pressure in the display screen.

Note that the blood pressure-related information display device according to a preferred embodiment of the present invention may substantially by configured only of the hospital terminal by further incorporating the functionality of the measurement unit 450 of the blood pressure meter 400 into the hospital terminal (into which the functionality of the server 300 has already been incorporated).

Furthermore, the control unit 310 of the server 300 may send only data to generate an image to the hospital terminal 200, rather than sending the image data, and the control unit 210 of the hospital terminal 200 may then generate the image itself.

Meanwhile, instead of using these types of hospital terminals (into which the functionality of the server 300 has been incorporated, and into which the measurement unit 450 of the blood pressure meter 400 has further been incorporated), the blood pressure-related information display device according to a preferred embodiment of the present invention may be configured using a smartphone or mobile telephone having the same functions and constituent elements of the respective types of hospital terminals.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

1-6. (canceled)

7. A blood pressure-related information display device that displays information related to a measurement subject's blood pressure in a display screen, the device comprising:
a blood pressure data obtainment unit configured to obtain blood pressure data of the measurement subject;
an evaluation result calculation unit configured to calculate, for a predetermined plurality of blood pressure evaluation items used to evaluate a state of the measurement subject's blood pressure, a blood pressure evaluation result indicating a quality of each of the plurality of blood pressure evaluation items by executing a statistical process on the blood pressure data measured on each day throughout a given period and evaluating a change in the blood pressure data over time; and
a display control unit configured or programmed to perform control to display a blood pressure evaluation item list enabling the plurality of blood pressure evaluation items to be viewed as a list in the display screen and displaying the calculated blood pressure evaluation results in association with and next to corresponding ones of the plurality of blood pressure evaluation items in the blood pressure evaluation item list.

8. The blood pressure-related information display device according to claim 7, wherein the blood pressure evaluation result is displayed as a mark that is distinguishable by color or by shape.

9. The blood pressure-related information display device according to claim 7, further comprising:
a comment storage unit configured to store a comment for the quality;
an item selection unit configured to select the blood pressure evaluation item for which the quality is displayed from the blood pressure evaluation item list; and
a comment display control unit configured or programmed to perform control to display, in the display screen, a comment regarding the quality of the selected blood pressure evaluation item by referring to the comment storage unit.

10. The blood pressure-related information display device according to claim 8, wherein the comment storage unit is configured to store the comment in accordance with content of the quality.

11. A non-transitory computer-readable medium including a computer program that causes a computer to execute a method for displaying information related to a measurement subject's blood pressure in a display screen, the method comprising:
a first step of obtaining blood pressure data of the measurement subject;
a second step of calculating, for a predetermined plurality of blood pressure evaluation items used to evaluate a state of the measurement subject's blood pressure, a blood pressure evaluation result indicating a quality of each of the blood pressure evaluation items, by executing a statistical process on the blood pressure data measured on each day throughout a given period and obtained in the first step and evaluating a change in the pressure data over time; and
a third step of displaying a blood pressure evaluation item list enabling the plurality of blood pressure evaluation items to be viewed as a list in the display screen and displaying the calculated blood pressure evaluation results in association with and next to corresponding ones of the plurality of blood pressure evaluation items in the displayed blood pressure evaluation item list.
